(19) United States
(12) Patent Application Publication
(10) Pub. No.: US 2022/0322917 A1
(43) Pub. Date: Oct. 13, 2022

(54) ENDOSCOPE PROCESSOR, ENDOSCOPE, AND ENDOSCOPE SYSTEM

(71) Applicant: HOYA CORPORATION, Tokyo (JP)

(72) Inventor: Nobuaki ABE, Tokyo (JP)

(73) Assignee: HOYA CORPORATION, Tokyo (JP)

(21) Appl. No.: 17/642,361

(22) PCT Filed: Jan. 26, 2021
(86) PCT No.: PCT/JP2021/002584

§ 371 Date: Mar. 11, 2022

(30) Foreign Application Priority Data
Mar. 10, 2020 (JP) 2020-041080

(51) Int. Cl. A61B 1/00; A61B 1/005

(52) U.S. Cl. CPC A61B 1/000096; A61B 1/00097; A61B 1/005

(57) ABSTRACT

A processor for an endoscope includes: an acquisition unit that acquires a detection value detected by an endoscope or a captured image captured by the endoscope; a specification unit that specifies operation information of a next stage based on the detection value or captured image acquired by the acquisition unit; and an output unit that outputs the operation information specified by the specification unit.

### TECHNICAL FIELD

The present technology relates to a processor for an endoscope, an endoscope, an endoscope system, an information processing method, a program, and a method for generating a learning model.

### BACKGROUND ART

An endoscope is a medical apparatus that enables observation and treatment of a desired place by being inserted into a body of a subject. An operator of the endoscope needs to perform an appropriate operation according to a shape state and an insertion position of the endoscope in the body of the subject. In the endoscope operation, in particular, a large intestine endoscope requires an advanced operation technique in order to insert the endoscope into a large intestine since a shape of the large intestine is complicated as compared with the shape of other internal organs subjected to an endoscopic examination. Therefore, a technique of observing the shape of the endoscope in the body and supporting the operation of the operator has been proposed. Patent Literature 1 discloses an insertion system capable of presenting, to the operator, an attention state related to an insertion operation, such as the shape of the endoscope.

### CITATION LIST

### Patent Literature

### Patent Literature 1: WO 2018/069992 A

### SUMMARY OF INVENTION

### Technical Problem

However, there is a problem that information for supporting the operation of the endoscope is not sufficient in Patent Literature 1.

An object of the present disclosure is to provide a processor for an endoscope or the like that outputs information for supporting an endoscope operation based on a state of the endoscope.

### Solution to Problem

A processor for an endoscope according to an aspect of the present disclosure includes: an acquisition unit that acquires a detection value detected by an endoscope or a captured image captured by the endoscope; a specification unit that specifies operation information of a next stage based on the detection value or captured image acquired by the acquisition unit; and an output unit that outputs the operation information specified by the specification unit.

### Advantageous Effects of Invention

According to the present disclosure, it is possible to output information for supporting the endoscope operation based on the state of the endoscope.

### BRIEF DESCRIPTION OF DRAWINGS

FIG. 1 is an explanatory view illustrating an appearance of an endoscope system.

FIG. 2 is an explanatory view for explaining a first example of a configuration of an insertion portion.

FIG. 3 is a cross-sectional view taken along line III-III illustrated in FIG. 2.

FIG. 4 is an explanatory view for explaining a second example of a configuration of an insertion portion.

FIG. 5 is an explanatory view for explaining a third example of a configuration of an insertion portion.

FIG. 6 is an explanatory view for explaining a fourth example of a configuration of an insertion portion.

FIG. 7 is an explanatory diagram for explaining a configuration of an endoscope system.

FIG. 8 is an explanatory diagram for explaining a configuration of a learning model of a first embodiment.

FIG. 9 is an explanatory diagram for explaining another configuration of a learning model.

FIG. 10 is a flowchart illustrating an example of a processing procedure of generating a learning model.

FIG. 11 is a flowchart illustrating an example of an operation support processing procedure using a learning model.

FIG. 12 is a diagram illustrating an example of a screen displayed on a display device.

FIG. 13 is an explanatory diagram for explaining an example of an icon of operation information.

FIG. 14 is an explanatory diagram for explaining a configuration of a learning model of a second embodiment.

FIG. 15 is a diagram illustrating an example of a screen displayed on a display device.

### DESCRIPTION OF EMBODIMENTS

The present invention will be specifically described with reference to the drawings illustrating embodiments of the invention.

### First Embodiment

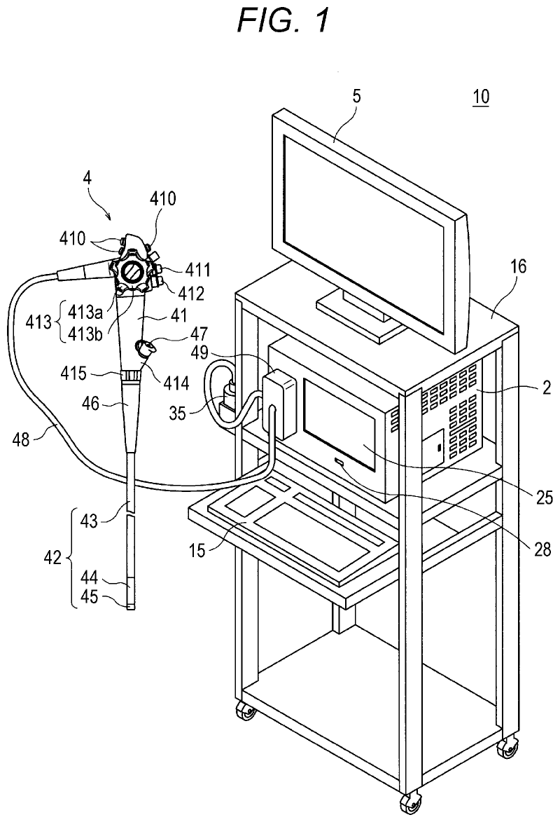

FIG. 1 is an explanatory diagram illustrating an appearance of an endoscope system 10. The endoscope system 10 according the first embodiment includes a processor 2 for an endoscope, and an endoscope 4. A display device 5 is connected to the processor 2 for an endoscope. The processor 2 for an endoscope, the endoscope 4, and the display device 5 are connected to each other via a connector, and transmit and receive an electric signal, a video signal, and the like.

The endoscope 4 is, for example, a large intestine endoscope for a lower digestive tract. The endoscope 4 is an instrument for performing diagnosis or treatment on a portion from a rectum to an end of a colon by inserting an insertion portion 42 having an image sensor at a distal end into an anus. The endoscope 4 transmits the electric signal of an observation target captured by the image sensor provided at the distal end to the processor 2 for an endoscope.

As illustrated in the drawing, the endoscope 4 includes an operation unit 41, an insertion portion 42, a universal cord 48, and a scope connector 49. The operation unit 41 is provided to be gripped by a user to perform various operations, and includes a control button 410, a suction button 411, an air/water supply button 412, an angulation knob 413, a channel inlet 414, and a hardness variable knob 415. The angulation knob 413 has a UD angulation knob 413a for a bending operation in an UP/DOWN (UD) direction and an RL angulation knob 413b for a bending operation in a RIGHT/LEFT (RL) direction. A forceps plug 47 having an insertion port for inserting a treatment tool or the like is fixed to the channel inlet 414.

The insertion portion 42 is a portion to be inserted into a luminal organ of a digestive tract of the subject, and includes a long soft portion 43 and a distal end portion 45 connected to one end of the soft portion 43 via a bending portion 44. The other end of the soft portion 43 is connected to the operation unit 41 via a bend preventing portion 46.

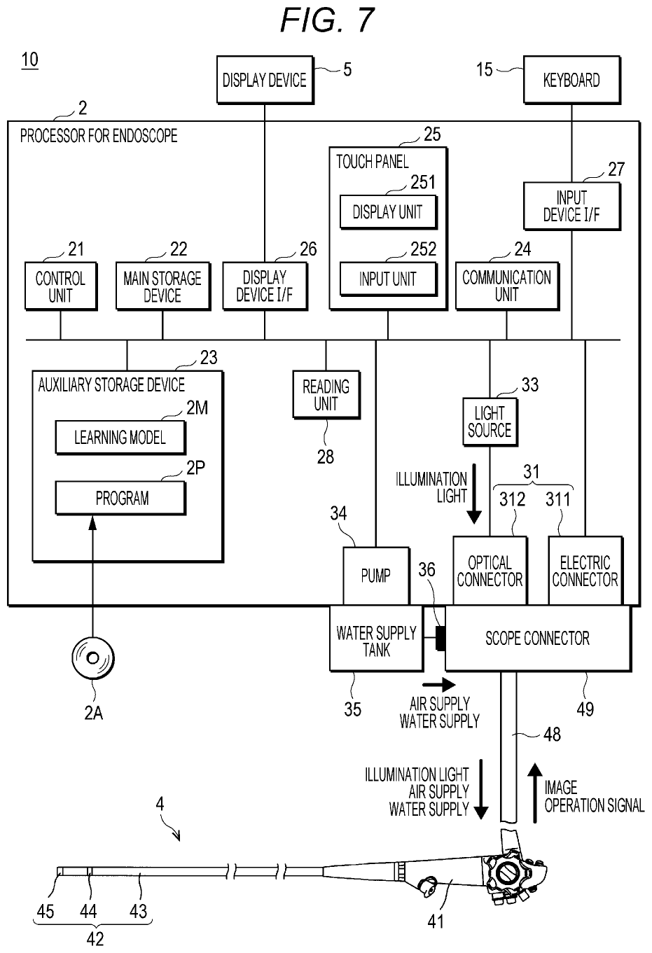

The universal cord 48 is flexible and long, has one end connected to the operation unit 41 and the other end connected to the scope connector 49. A fiber bundle, a cable bundle, an air supply tube, a water supply tube, and the like are inserted inside the scope connector 49, the universal cord 48, the operation unit 41, and the insertion portion 42. The scope connector 49 is provided with an air/water supply metal port 36 (see FIG. 7) for connecting an air/water supply tube.

The processor 2 for an endoscope is an information processing device that performs image processing on a captured image captured by the image sensor provided at the distal end of the endoscope 4, generates an endoscopic image, and outputs the endoscopic image to the display device 5. The processor 2 for an endoscope has a substantially rectangular parallelepiped shape and includes a touch panel 25 provided on one surface thereof. A reading unit 28 is disposed at a lower portion of the touch panel 25. The reading unit 28 is a connection interface for performing reading and writing on a portable recording medium such as a USB connector, a secure digital (SD) card slot, a compact disc read only memory (CD-ROM) drive, or the like.

The display device 5 is a liquid crystal display device or an organic electro luminescence (EL) display device, and displays the endoscopic image or the like output from the processor 2 for an endoscope. The display device 5 is installed on an upper stage of a storage shelf 16 with casters. The processor 2 for an endoscope is stored in a middle stage of the storage shelf 16. The storage shelf 16 is disposed in the vicinity of a bed for an endoscopic examination (not illustrated). The storage shelf 16 includes a pull-out shelf on which a keyboard 15 connected to the processor 2 for an endoscope is mounted.

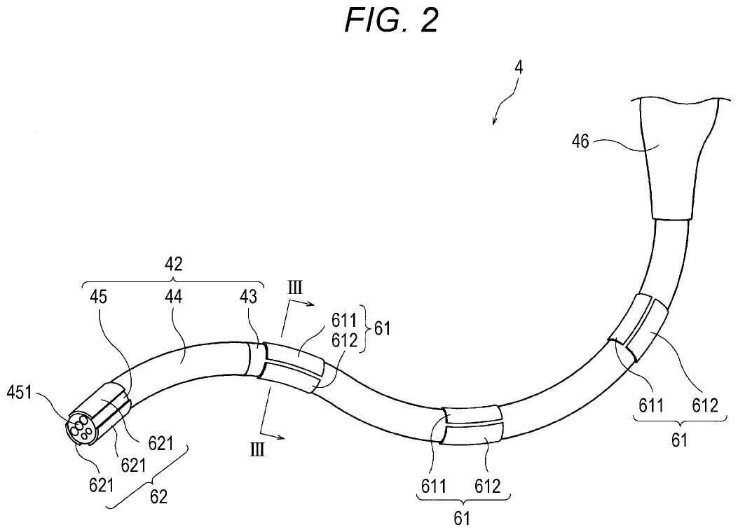

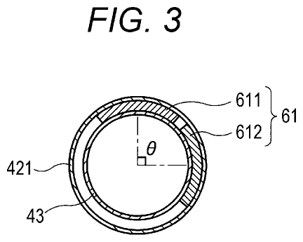

FIG. 2 is an explanatory view for explaining a first example of a configuration of the insertion portion 42. FIG. 3 is a cross-sectional view taken along line III-III illustrated in FIG. 2. The insertion portion 42 has a long tubular shape coated with a sheath (outer sheath) 421 made of a resin material, and includes the soft portion 43, the bending portion 44, and the distal end portion 45 as described above. FIG. 2 illustrates a state in which the sheath 421 is removed.

The soft portion 43 has flexibility, and is inserted into the body while being bent so as to be suitable for a bent situation in an intestinal tract by an external force. In the soft portion 43, a coil (not illustrated) installed inside the soft portion 43 expands and contracts and the hardness thereof is changed by operating the hardness variable knob 415 according to the situation in the intestinal tract. The hardness is variable in four stages of, for example, 1 to 4, and the larger a numerical value, the higher the hardness.

As illustrated in FIG. 2, the soft portion 43 is provided with one or a plurality of strain sensor units 61 on an outer circumference as state detection means for detecting the state, shape, and the like of the insertion portion 42. In the example of FIG. 2, three strain sensor units 61 are provided. Each of the strain sensor units 61 is disposed at regular intervals (for example, 15 cm to 20 cm) in a longitudinal direction. One strain sensor unit 61 includes a first strain sensor 611 and a second strain sensor 612. As illustrated in FIG. 3, on the same circumference of the outer circumference of the soft portion 43, the first strain sensor 611 and the second strain sensor 612 are disposed at positions separated by a central angle θ. The central angle θ is approximately 90 degrees. Each of the first strain sensor 611 and the second strain sensor 612 is fixed to the soft portion 43 by, for example, an adhesive or a tacky adhesive. The first strain sensor 611 and the second strain sensor 612 output signals indicating the strain of the soft portion 43 according to the external force. The first strain sensor 611 and the second strain sensor 612 are disposed so as to be orthogonal to each other with respect to the center of the soft portion 43, so that it is possible to detect a strain amount of the soft portion 43 in a vertical direction and a horizontal direction.

An operation wire (not illustrated) is disposed inside the bending portion 44 and the soft portion 43. The bending portion 44 performs the bending operation in the UD direction and the RL direction of the endoscope by pulling the operation wire in conjunction with the operation of the angulation knob 413. The direction of the distal end portion 45 is changed according to the bending operation of the bending portion 44.

The distal end portion 45 is formed of a housing made of a resin having hardness. A distal end surface of the distal end portion 45 is provided with an observation window 451 for acquiring an image of the observation target, an illumination window for irradiating the observation target with illumination light, an air/water supply nozzle for supplying air and water, a forceps outlet connected to the channel inlet 414, and the like. An imaging unit (not illustrated) including an image sensor such as a charge coupled device (CCD) image sensor and an objective optical system for image formation is built in the depth of the observation window 451. The image sensor receives light reflected from an object through the observation window 451 and performs photoelectric conversion. An electric signal generated by the photoelectric conversion is subjected to signal processing such as A/D conversion and noise removal by using a signal processing circuit (not illustrated), and is output to the processor 2 for an endoscope.

As illustrated in FIG. 2, a pressure sensor unit 62 is further provided as state detection means on a side surface of the distal end portion 45. The pressure sensor unit 62 includes one or a plurality of pressure sensors 621. In the example of FIG. 2, three pressure sensors 621 are disposed at equal intervals on the outer circumference of the distal end portion 45. Each of the pressure sensors 621 outputs a signal indicating the pressure of the distal end portion 45 by contacting with the intestinal wall or the like in the body. The pressure sensor 621 is fixed to the distal end portion 45 by, for example, an adhesive or a tacky adhesive.

Each of the first strain sensor 611, the second strain sensor 612, and the pressure sensor 621 includes signal lines (not illustrated) electrically connected to each sensor. Each of the signal lines extends along the outer circumference of the soft portion 43, passes through the inside of the operation unit 41 and the universal cord 48, and is connected to the processor 2 for an endoscope via the scope connector 49. The detection values obtained by the various sensors are transmitted through the signal lines and output to the processor 2 for an endoscope via the signal processing circuit (not illustrated). Each of the signal lines may be fixed to the soft portion 43 by, for example, an adhesive or a tacky adhesive, and may be fixed to the soft portion 43 by the sheath 421.

In the endoscope system 10, the state, shape, and the like of the insertion portion 42 in the intestinal tract of the subject are detected by the state detection means using the various sensors described above. The state, shape, and the like of the insertion portion 42, that is, strain and pressure of the insertion portion 42 become pressure applied to the intestinal tract of the subject, which causes a pain given to the subject at the time of the endoscopic examination. By detecting the state, shape, and the like of the insertion portion 42 with a sensor and quantifying the state, shape, and the like, accurate determination can be made regardless of experience and ability of the operator. The processor 2 for an endoscope provides the operator with optimum operation information of the next stage estimated by a learning model 2M to be described later according to the detection result, and thus the smooth endoscope operation of the operator is supported.

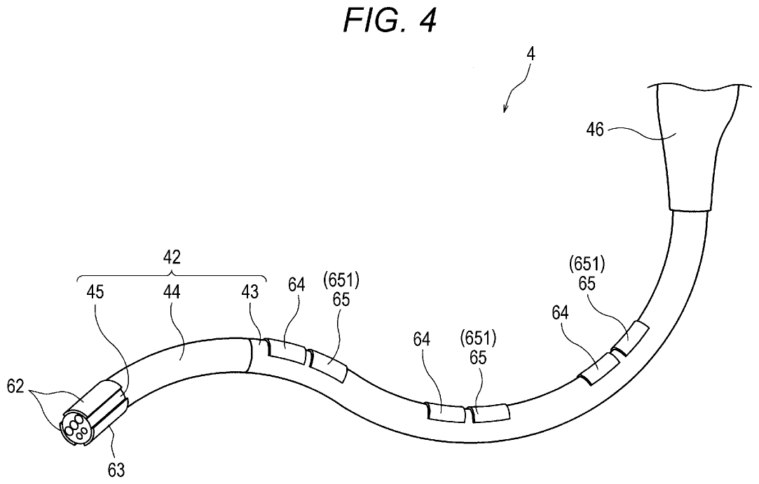

In the above description, the example in which the strain sensor unit 61 and the pressure sensor unit 62 are provided as the state detection means has been described, but the detection means such as the state and shape, and the like of the insertion portion 42 are not limited, and other sensors may be used. FIG. 4 is an explanatory view for explaining a second example of a configuration of the insertion portion 42. In the example illustrated in FIG. 4, the insertion portion 42 includes an acceleration sensor 63, an angle sensor 64, and a magnetic sensor 65 as the state detection means instead of the strain sensor unit 61 described in the example of FIG. 2.

The acceleration sensor 63 is disposed on the outer circumference of the distal end portion 45. The acceleration sensor 63 outputs a signal indicating the acceleration of the distal end portion 45 corresponding to the insertion operation of the insertion portion 42. The angle sensor 64 is disposed on the outer circumference of the soft portion 43. A plurality of the angle sensors 64 may be provided, and in this case, may be disposed at predetermined intervals in the longitudinal direction of the soft portion 43. Each of the angle sensors 64 has a coordinate system having an X-axis, a Y-axis, and a Z-axis that respectively coincide with the horizontal direction, the longitudinal direction, and the vertical direction of the insertion portion 42 with the center of the angle sensor 64 as the origin. Each of the angle sensors 64 outputs a signal indicating a yaw, a roll, and a pitch in three axes directions in the coordinate system. The magnetic sensor 65 includes a magnetic coil 651, and is disposed on the outer circumference of the soft portion 43. A plurality of the magnetic sensors 65 may be provided, and in this case, may be disposed at predetermined intervals in the longitudinal direction of the soft portion 43. Each of the magnetic coils 651, which is the magnetic sensor 65, outputs a magnetic field signal. The magnetic field signal generated from the magnetic coil 651 is received by an external reception device communicably connected to the processor 2 for an endoscope and transmitted to the processor 2 for an endoscope. A position, shape, and the like of the endoscope 4 are derived based on magnitude of the magnetic field, and the output acceleration and angle. Note that the endoscope system 10 may use these other sensors and one or a plurality of sensors selected from the strain sensor and the pressure sensor described above in combination.

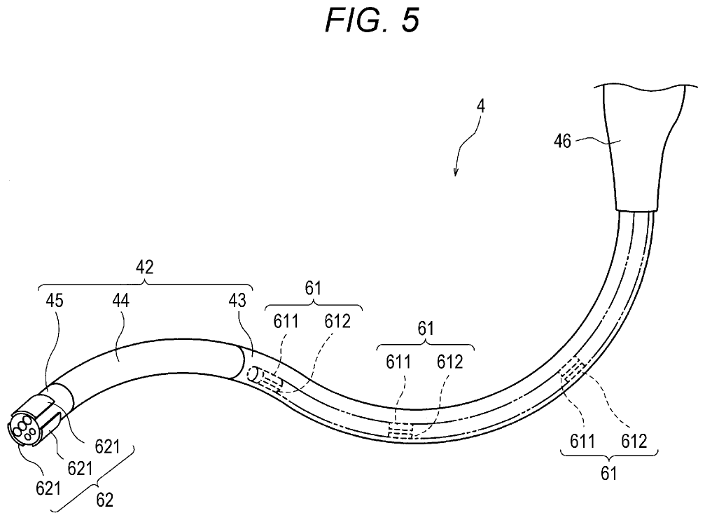

Note that the various sensors described above are not limited to those provided on the outer circumference of the insertion portion 42. FIG. 5 is an explanatory view for explaining a third example of a configuration of the insertion portion 42. For example, as illustrated in FIG. 5, the strain sensor unit 61 may be disposed inside the soft portion 43. The strain sensor unit 61 may be provided, for example, on the outer circumference of a fiber bundle, a cable bundle, or the like inserted into the insertion portion 42. As described above, the diameter of the insertion portion 42 can be reduced by providing the various sensors inside the insertion portion 42.

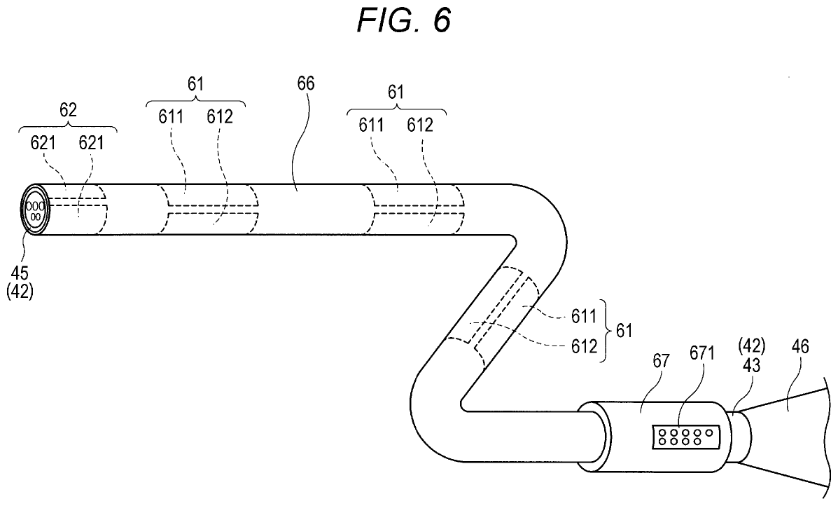

Furthermore, the various sensors are not limited to the example of being formed integrally with the insertion portion 42. The various sensors may be formed in a detachable manner and attached to the insertion portion 42. FIG. 6 is an explanatory view for explaining a fourth example of a configuration of the insertion portion 42. The various sensors are incorporated in a tube 66 that is a cylindrical external member. FIG. 6 illustrates an example in which the strain sensor unit 61 and the pressure sensor unit 62 are incorporated and disposed in the tube 66. The tube 66 is attachable to or detachable from the insertion portion 42, and the various sensors are disposed by attaching the tube 66 to the outer circumference of the insertion portion 42. One end of the tube 66 is fixed to the distal end side of the endoscope 4. A tube connector 67 including an external connection unit 671 is provided at the other end of the tube 66. The tube 66 is connected to an endoscope connector 31 (see FIG. 7) of the processor 2 for an endoscope via a connection cable (not illustrated) connected to the external connection unit 671. Detection values of the various sensors are output to the processor 2 for an endoscope through a signal line extended to the external connection unit 671. Note that the tube 66 may be connected to an external information processing device via a connection cable (not illustrated) connected to the external connection unit 671. In this case, the detection values of the various sensors are acquired by the external information processing device and transmitted to the processor 2 for an endoscope.

Note that the various sensors configured to be attachable or detachable may be provided, for example, on a probe or the like that can be inserted into a channel from the channel inlet 414, and may be disposed inside the insertion portion 42.

FIG. 7 is an explanatory diagram for explaining a configuration of the endoscope system 10. As described above, the endoscope system 10 includes the processor 2 for an endoscope and the endoscope 4.

The processor 2 for an endoscope includes a control unit 21, a main storage device 22, an auxiliary storage device 23, a communication unit 24, a touch panel 25, a display device interface (I/F) 26, an input device I/F 27, a reading unit 28, an endoscope connector 31, a light source 33, a pump 34, and a bus. The endoscope connector 31 includes an electric connector 311 and an optical connector 312.

The control unit 21 includes an arithmetic processing device such as a central processing unit (CPU), a micro-processing unit (MPU), or a graphics processing unit (GPU). The control unit 21 executes processing by using a memory such as a built-in read only memory (ROM) or a built-in random access memory (RAM) and controlling configuration units. The control unit 21 is connected to hardware units constituting the processor 2 for an endoscope via the bus. The control unit 21 executes various computer programs stored in the auxiliary storage device 23 to be described later, controls the operation of each hardware unit, and thus realizes a function as the processor 2 for an endoscope in the present embodiment. The control unit 21 is described as a single processor in FIG. 7, but the control unit 21 may be a multiprocessor.

The main storage device 22 is a storage device such as a static random access memory (SRAM), a dynamic random access memory (DRAM), or a flash memory. The main storage device 22 temporarily stores information necessary in the middle of processing performed by the control unit 21 and a program being executed by the control unit 21.

The auxiliary storage device 23 is a storage device such as an SRAM, a flash memory, or a hard disk. The auxiliary storage device 23 stores a program 2P executed by the control unit 21 and various data necessary for executing the program 2P. The auxiliary storage device 23 further stores a learning model 2M. The learning model 2M is an identifier that identifies information for supporting the operation of the endoscope, and is a learning model generated by machine learning. The learning model 2M is defined by definition information. The definition information of the learning model 2M includes, for example, structure information and layer information of the learning model 2M, channel information of each layer, and learned parameters. The auxiliary storage device 23 further stores the definition information related to the learning model 2M.

The program 2P stored in the auxiliary storage device 23 may be a program read from a recording medium 2A capable of being read by the control unit 21. The recording medium 2A is, for example, a portable memory such as a CD-ROM, a USB memory, an SD card, a micro SD card, or a CompactFlash (registered trademark). Furthermore, the program 2P may be a program downloaded from an external computer (not illustrated) connected to a communication network (not illustrated), and be stored in the auxiliary storage device 23. Note that the auxiliary storage device 23 may be configured by a plurality of storage devices, or may be an external storage device connected to the processor 2 for an endoscope.

The communication unit 24 is an interface for data communication between the processor 2 for an endoscope and the network (not illustrated). The touch panel 25 includes a display unit 251 such as a liquid crystal display panel, and an input unit 252 layered on the display unit 251.

The display device I/F 26 is an interface for connecting the processor 2 for an endoscope and the display device 5. The input device I/F 27 is an interface for connecting the processor 2 for an endoscope and an input device such as a keyboard 15.

The light source 33 is a high-luminance white light source such as a light emitting diode (LED) or a xenon lamp. The light source 33 is connected to the bus via a driver (not illustrated). Turning on and off of the light source 33 and change of luminance are controlled by the control unit 21. The illumination light emitted from the light source 33 is incident on the optical connector 312. The optical connector 312 engages with the scope connector 49 to supply the illumination light to the endoscope 4.

The pump 34 generates pressure for an air/water supply function of the endoscope 4. The pump 34 is connected to the bus via the driver (not illustrated). Turning on and off of the pump 34 and change of pressure are controlled by the control unit 21. The pump 34 is connected to the air/water supply metal port 36 provided in the scope connector 49 via a water supply tank 35.

Note, in the present embodiment, the processor 2 for an endoscope will be described as one information processing device, but processing may be performed by distributing the processor 2 for an endoscope into a plurality of processors, or the processor may be configured by a virtual machine.

The function of the endoscope 4 connected to the processor 2 for an endoscope will be outlined. The illumination light emitted from the light source 33 is radiated from the illumination window provided at the distal end portion 45 via the optical connector 312 and the fiber bundle inserted into the endoscope 4. A range illuminated by the illumination light is captured as an image by the image sensor provided at the distal end portion 45. The captured image is transmitted from the image sensor to the processor 2 for an endoscope via the cable bundle and the electric connector 311. The captured image subjected to the image processing by the processor 2 for an endoscope is displayed on the display device 5 or the display unit 251.

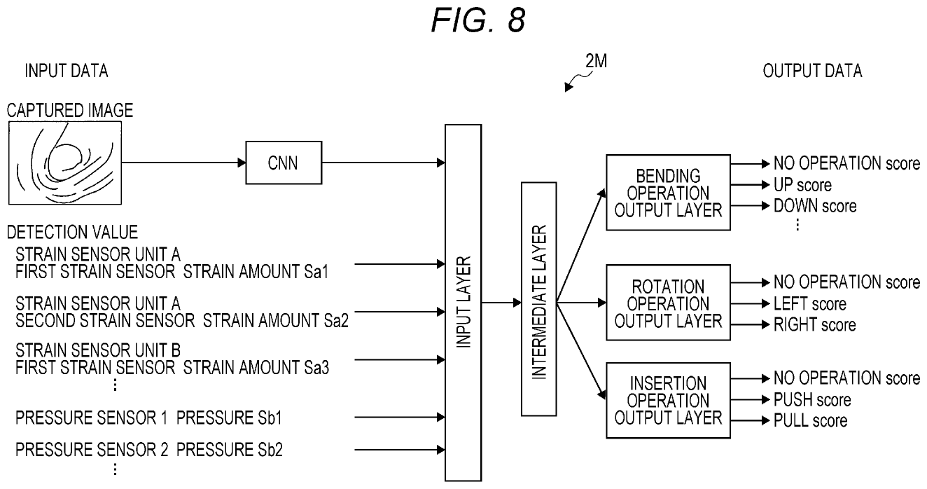

FIG. 8 is an explanatory diagram for explaining a configuration of the learning model 2M of the first embodiment. The learning model 2M is generated and trained by deep learning using a neural network. The learning model 2M of the first embodiment is, for example, a convolution neural network (CNN). In the example illustrated in FIG. 8, the learning model 2M includes an input layer that receives the captured image and the detection value, an output layer that outputs the operation information of the next stage, and an intermediate layer (hidden layer) that extracts feature amounts of the captured image and detection value. The intermediate layer includes a plurality of channels through which the feature amounts of the captured image and detection value, and passes the feature amounts extracted using various parameters to the output layer. The intermediate layer may include a convolution layer, a pooling layer, a fully-connected layer, and the like.

Input data input to the input layer of the learning model 2M are the captured image and the detection value at the same time point. The input captured image may be a captured image itself captured by the image sensor, or may be an endoscopic image that is made to be easily seen by the operator by performing various image processing such as gamma correction, white balance correction, and shading correction on the captured image. The captured image may be an image in the middle of generating the endoscopic image from the captured image. The captured image may be an image obtained by further performing various image processing such as reduction processing and averaging on the endoscopic image. The input captured image is, for example, a still image obtained by cutting out one frame from a moving image. The captured image may be a still image captured at appropriate timing separately from the moving image. Note that the input captured image may be a plurality of pieces of data acquired in a time-series manner. The captured image may be an image in which feature data extracted via a network including a convolution layer is input to the learning model 2M.

Moreover, each of the detection values obtained by various sensors provided on the insertion portion 42 is vectorized and input to the input layer. In the present embodiment, the detection value includes a detection value of a strain amount obtained by the strain sensor unit 61 and a detection value of pressure obtained by the pressure sensor unit 62. Specifically, the input data includes strain amounts in the vertical direction and the horizontal direction at each position of the bending portion 44, obtained by a plurality of the first strain sensors 611 and a plurality of the second strain sensors 612 provided on the bending portion 44, and detection values of pressure in each direction of the outer circumference of the distal end portion 45, obtained by a plurality of the pressure sensors 621 provided on the distal end portion 45. Identification information of a sensor may be input to the input layer in association with the detection value, the identification information including an arrangement place or the like of each sensor. The detection value may be input as an image obtained by graphing data at a plurality of time points stored in a time series manner. The detection value may be input as an image obtained by graphing data subjected to frequency conversion at the detection time point.

The output data output from the output layer of the learning model 2M is operation information of the next stage. The operation information is information regarding the operation of the insertion portion 42 of the endoscope 4, and may include, for example, an operation direction of each operation of bending, rotation, and insertion. In the present embodiment, the learning model 2M includes a plurality of output layers that output information indicating the bending operation, the rotation operation, and the insertion operation, respectively. The bending operation corresponds to an operation of the angulation knob 413, and includes, for example, an operation direction of UP, DOWN, RIGHT, and LEFT and no operation (maintaining the current state). The rotation operation is an operation of twisting the insertion portion 42, and includes, for example, right and left operation directions and no operation (maintaining the current state). The insertion operation is an insertion operation of the insertion portion 42, and includes, for example, forward and backward directions such as pushing and pulling directions and no operation (maintaining the current state). As the operation information of the next stage, optimum information is estimated based on the state, shape, and the like of the insertion portion 42 according to the endoscopic image and the detection value. For example, in a case where the detection amount of the pressure sensor 621 located in any place of the bending portion 44 is great, the bending operation indicating a direction in which the pressure is lowered is output according to the arrangement place of the pressure sensor 621. In a case where the detection amount of the strain sensor is great, the operation information for reducing the strain is output according to the arrangement place of the strain sensor.

The output layer includes channels each corresponding to the set operation information, and outputs accuracy for each operation information as a score. The processor 2 for an endoscope can set the operation information having the highest score or the operation information having a score equal to or greater than a threshold as an output value of the output layer. Note that the output layer may have one output channel that outputs the most accurate operation information instead of having a plurality of output channels that output the accuracy of each operation information. As described above, in a case where the captured image and the detection value are input, the learning model 2M outputs the operation information of the next stage.

The output operation information is not limited to the above example. For example, the operation information may include a hardness variable operation of the soft portion 43. The hardness variable operation corresponds to the operation of the hardness variable knob 415, and is indicated by, for example, numerical values of 1 to 4 corresponding to the setting of the hardness variable knob 415 and no operation (maintaining the current state). The operation information may include operation information regarding air supply, suction, or the like from the other distal end portion 45. An air supply operation and a suction operation correspond to operations of the air/water supply button 412 and the suction button 411, respectively, and are indicated with, for example, operation or no operation. The air supply operation and the suction operation may be output with information such as an operation time and an operation amount.

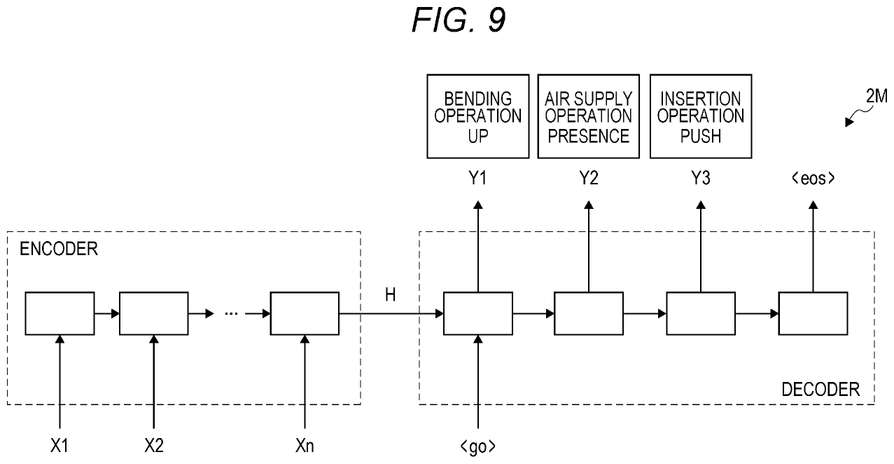

Although the example in which the learning model 2M is CNN has been described above, the learning model 2M is not limited to the CNN. In a case where time-series data is acquired, a neural network other than the CNN, for example, a recurrent neural network (RNN) or a long short term memory (LSTM) network may be used. FIG. 9 is an explanatory diagram for explaining another configuration of the learning model 2M.

In the example illustrated in FIG. 9, the learning model 2M is a Sequence to Sequence (Seq2Seq) model using the LSTM. The Seq2Seq includes an encoder and a decoder, and can output an output sequence having an arbitrary length from an input sequence having an arbitrary length. In the example of FIG. 9, the learning model 2M is configured to output the time series data indicating the operation information in a case where the time-series data of the captured image and the detection value are input.

The encoder extracts features of the input data. Although the encoder is described as a single block in FIG. 9, the encoder has an input layer and an intermediate layer (hidden layer). Time-series data X1, X2, . . . , Xn of the captured image and the detection value are sequentially input to the encoder. The previous output of the intermediate layer is input to the intermediate layer in addition to the output from the input layer. The encoder outputs feature information H indicating features of the input captured image and detection value.

The decoder outputs operation information of a plurality of stages. Although the decoder is described as a single block in FIG. 9, the decoder has an intermediate layer (hidden layer) and an output layer. The feature information H output from the encoder is input to the decoder. When <go> instructing start of output is input to the decoder and calculation is executed, output data Y1, Y2, . . . , and Ym indicating operation information are sequentially output from the output layer. <eos> indicates the end of output. The output data Y1, Y2, . . . , and Ym represent time-series data indicating the operation information. In the example of FIG. 9, the time-series data Y1, Y2, and Y3 indicating three pieces of operation information are output from the output layer. Y1 indicates the bending operation UP which is the operation information at time t1, Y2 indicates the presence of the air supply operation which is the operation information at time t2, and Y3 indicates the insertion operation of pushing which is the operation information at time t3. As described above, in a case where the time-series data of the captured image and the detection value are input, the learning model 2M outputs prediction of the operation information of a plurality of stages.

Note that the learning model 2M is not limited to a learning model using the neural network described in the above example. The learning model 2M may be a model trained by another algorithm such as a support vector machine or a regression tree.

The learning model 2M described above is generated in a learning phase that is a previous stage of an operation phase of performing operation support, and the generated learning model 2M is stored in the processor 2 for an endoscope.

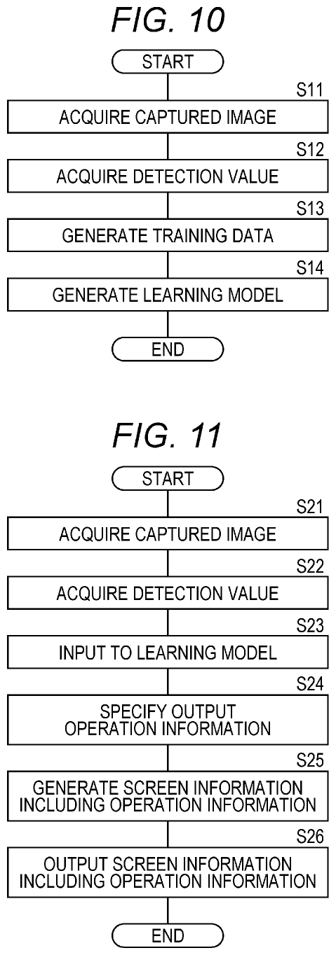

FIG. 10 is a flowchart illustrating an example of a processing procedure of generating the learning model 2M. The control unit 21 of the processor 2 for an endoscope executes the following processing in the learning phase.

The control unit 21 acquires a captured image captured by the image sensor provided at the distal end portion 45 of the endoscope 4 (Step S11). The captured image is obtained as, for example, a moving image, and is constituted by still images of a plurality of frames such as 60 frames per second. The control unit 21 executes various image processing on the captured image as necessary

Next, the control unit 21 acquires the detection value of various sensors from the endoscope 4 (Step S12). Specifically, the control unit 21 acquires the detection value of each of the first strain sensor 611, the second strain sensor 612, and the pressure sensor 621. The control unit 21 may acquire identification information of each sensor, a detection time point of the detection value, and the like in association with the detection value of each sensor. The control unit 21 temporarily stores the captured image and detection value at the same time point in association with each other in the auxiliary storage device 23.

The control unit 21 generates training data obtained by annotating the operation information of the next stage to the captured image and detection value at the same time point with reference to the auxiliary storage device 23 (Step S13). The training data is, for example, a data set obtained by annotating operation information of a skilled operator in the next stage, as a correct value, to the captured image and the detection value. The control unit 21 generates multiple training data causing the operation information to be associated with the captured image and the detection value at each time point.

The operation information of the skilled operator may be acquired, for example, by capturing an operation state of the skilled operator with one or a plurality of imaging devices and analyzing the captured image. Information regarding each operation of the operator, such as bending, rotation, insertion, air supply, suction, or hardness variable, is acquired based on the image analysis. Furthermore, the operation information may be acquired using various sensors provided in the endoscope operated by the skilled operator. For example, the acceleration sensor, the angle sensor, the pressure sensor, and the like are provided on each operation button, the insertion portion, and the like, and the operation of each operation button and the operation of the entire insertion portion are detected using these sensors. Furthermore, the operation information may be acquired by performing image analysis on the captured image captured by the endoscope operated by the skilled operator. The control unit 21 collects a large amount of test data and operation information, and accumulates the training data generated based on the collected data in a database (not illustrated) of the auxiliary storage device 23.

By using the generated training data, the control unit 21 generates the learning model 2M that outputs the operation information of the next stage in a case where the captured image and the detection value are input (Step S14). Specifically, the control unit 21 accesses the database of the auxiliary storage device 23 and acquires a set of training data used for generating the learning model 2M. The control unit 21 inputs the captured image and the detection value at a predetermined time, included in the training data, to the input layer of the learning model 2M, and acquires a prediction value of the operation information of the next stage from the output layer of the learning model 2M. An initial setting value is given to the definition information describing the learning model 2M at the stage before the learning is started. The control unit 21 compares the prediction value of the operation information with the operation information that is the correct value, and learns parameters, weights, and the like in the intermediate layer so as to reduce the difference. When the learning is completed with the magnitude of the difference and the number of times of the learning, which satisfies predetermined criteria, optimized parameters are obtained. The control unit 21 stores the generated learning model 2M in the auxiliary storage device 23, and ends the series of processing.

The example in which the control unit 21 of the processor 2 for an endoscope executes a series of processing has been described above, but the present embodiment is not limited to this. A part or all of the above processing may be executed by the external information processing device (not illustrated) communicably connected to the processor 2 for an endoscope. The processor 2 for an endoscope and the information processing device may perform, for example, a series of processing in cooperation by performing communication between processes. The control unit 21 of the processor 2 for an endoscope may only transmit the captured image captured by the image sensor and the detection value detected by the sensor, and the information processing device may perform subsequent processing. Furthermore, the learning model 2M may be generated by the information processing device and trained by the processor 2 for an endoscope.

Using the learning model 2M generated as described above, in the endoscope system 10, optimal operation information according to the operation state is provided. Hereinafter, a processing procedure executed by the processor 2 for an endoscope in the operation phase will be described.

FIG. 11 is a flowchart illustrating an example of an operation support processing procedure using the learning model 2M. The control unit 21 of the processor 2 for an endoscope executes the following processing at timing after training of the learning model 2M is completed. The control unit 21 may execute the following processing each time the endoscope 4 is operated, and for example, may execute the following processing only in a case where a start request for operation support is received based on input contents from the input unit 252 or the like connected to the device itself.

An operation of the operator is started, and imaging with the endoscope 4 is started. The control unit 21 acquires the captured image from the endoscope 4 in real time (Step S21), and generates an endoscopic image obtained by performing predetermined image processing on the acquired captured image. Next, the control unit 21 acquires the detection value at the time point of imaging from the endoscope 4 (Step S22). Specifically, the control unit 21 acquires the detection value of each of the first strain sensor 611, the second strain sensor 612, and the pressure sensor 621. The control unit 21 may acquire identification information of each sensor, a detection time point of the detection value, and the like in association with the detection value of each sensor. The control unit 21 temporarily stores the acquired captured image and the acquired detection value in the auxiliary storage device 23.

The control unit 21 inputs the captured image and the detection value to the learning model 2M (Step S23). The captured image input to the learning model may be an endoscopic image or an image obtained by performing predetermined image processing on the captured image or the endoscopic image. The control unit 21 specifies the operation information output from the learning model 2M (Step S24).

The control unit 21 generates screen information including the operation information of the next stage based on the specified operation information (Step S25). The control unit 21 outputs the screen information including the generated operation information by using the display device 5 (Step S26), and ends the series of processing. Note that after executing the processing of Step S26, the control unit 21 may perform loop processing to execute the processing of Step S21 again. As described above, the processor 2 for an endoscope generates the optimum operation information of the next stage based on the endoscopic image indicating the state of the endoscope 4 and the detection value, displays the generated operation information on the display device 5, and thus supports smooth operation of the endoscope 4 by the operator.

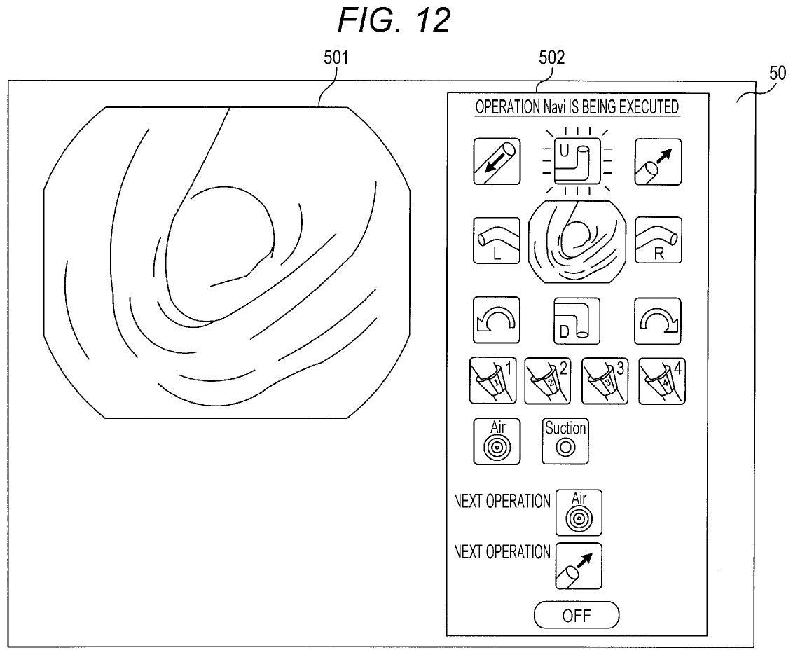

FIG. 12 is a diagram illustrating an example of a screen displayed on the display device 5. The display device 5 displays an operation information screen 50 based on the screen information. On the operation information screen 50, an endoscopic image 501 and a navigation image 502 including the operation information of the next stage are displayed in parallel. In the example of FIG. 12, the operation information of the next stage is displayed on the navigation image 502 as iconified information. Display or Non-display of the navigation image 502 can be selected by a switching button on the navigation image 502. An icon indicating each operation information may be disposed on the navigation image 502, for example, at a position corresponding to the operation content with the endoscopic image as the center. In the example of FIG. 12, icons indicating the bending operations UP, DOWN, RIGHT, and LEFT are disposed on the upper side, the lower side, the right side, and the left side of the endoscopic image. Note that each icon may be superimposed and displayed on the endoscopic image 501.

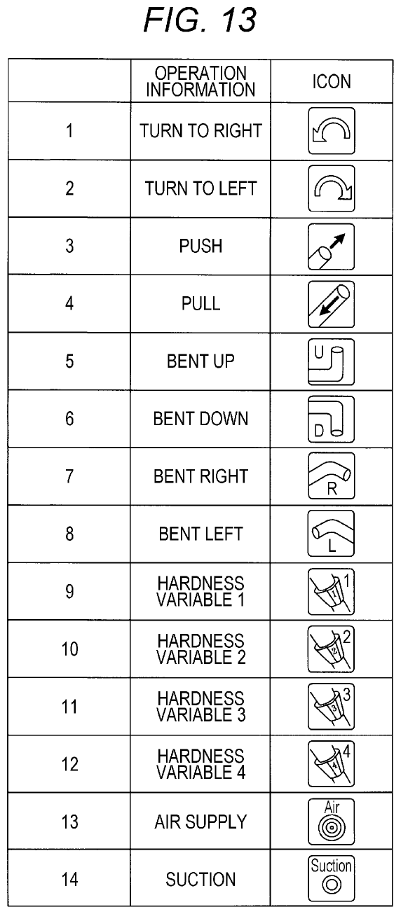

FIG. 13 is an explanatory diagram for explaining an example of the icon of the operation information. The operation information of the next stage is displayed using the icon illustrated in a mode in which each operation content can be easily recognized. As illustrated in FIG. 13, for example, the bending operation is displayed with icons indicating insertion portions bent upward, downward, rightward, and leftward, which correspond to the operation contents of the UD angulation knob 413a and the RL angulation knob 413b. The rotation operation and the insertion operation are displayed with icons using arrows indicating right and left rotation directions or forward and backward directions. As other operation information, the hardness variable operation is displayed with icons including the hardness variable knob 415 and set values (for example, 1 to 4) of the hardness variable knob 415. The air supply operation and the suction operation are displayed with icons respectively including characters such as “Air” and “Suction” or illustrations, which indicate the respective operation contents. The icons respectively indicating the respective air supply operation and the suction operation may be generated with the character or illustration corresponding to operation time and operation amount. The control unit 21 stores a table in which the operation information and the display content of the icon are associated with each other.

In a case where the operation information output by the learning model 2M is specified, the control unit 21 performs image processing such as turning on the icon or changing the color of the icon of the specified operation information, and then displays the navigation image 502 obtained after the image processing on the display device 5. As the operation information of the next stage, the control unit 21 may display only the operation information with the highest accuracy as the output information, or may display, as the output information, a predetermined plurality of pieces of operation information (for example, 3 pieces) in descending order of accuracy. The control unit 21 may display a plurality of pieces of operation information having an accuracy of a predetermined threshold or greater as output information. FIG. 13 illustrates an example in which the icon of the operation information as the output information is turned on as highlighting of the operation information, but other methods may be used. For example, the color, size, shape, blinking/turning on, display state of the icon, or a combination thereof may be changed to realize the highlighting. Furthermore, the control unit 21 may display only the icon indicating the operation information of the next stage on the display device 5, for example, instead of highlighting the operation information of the next stage. The control unit 21 generates the navigation image including the icon corresponding to the specified operation information, and displays the navigation image on the display device 5 with reference to the table (not illustrated) storing the operation information and the icon in association with each other.

In a case where prediction of the operation information in a plurality of stages in time series is acquired by the learning model 2M, the operation information screen 50 including a plurality of pieces of the operation information may be displayed. In the example of the operation information screen 50 illustrated in FIG. 12, the control unit 21 acquires the time-series data Y1 (bending operation UP), the time-series data Y2 (presence of air supply operation), and the time-series data Y3 (insertion operation is pressed), which are output from the learning model 2M. The control unit 21 reads a display mode of the icon corresponding to each output information from the table, performs image processing according to the display mode of the read icon, and then displays the navigation image 502 obtained after the image processing on the display device 5. In the navigation image 502, as illustrated in FIG. 12, in addition to the operation information Y1 of the next stage, icons indicating the operation information Y2 and operation information Y3 in a plurality of stages in time series are displayed including an operation order.

In this manner, the operation information is displayed together with the endoscopic image by using the icon with which the operation content is easily recognized. The operator can instantaneously recognize the operation information without moving a visual line from the display device 5. Note that the operation information is not limited to information that is iconified and output. The control unit 21 may display the operation information with a character or the like, or may notify the operation information by voice or the like via a speaker (not illustrated).

According to the present embodiment, it is possible to provide the operation information for navigating next operation content in real time at the time of the endoscope operation. As the operation information, optimal operation information is output by the learning model 2M according to an image captured by the endoscope 4 and a sensor detection value. Since the endoscope operation can be smoothly performed based on the optimum operation information, for example, even the operator with a low skill level can perform an endoscopic examination in a short time. Moreover, by referring to the navigation content provided as the operation information, it is possible to prevent erroneous operation and reduce the possibility of causing pain to the subject.

### Second Embodiment

In the second embodiment, a configuration of estimating operation information and information regarding a position of the endoscope 4 by using a learning model will be described. Hereinafter, a difference between the second embodiment and the first embodiment will be described. Since the other configurations except a configuration to be described later are similar to those of the first embodiment, the same reference numerals are given to the common configurations, and the detailed description thereof will be omitted.

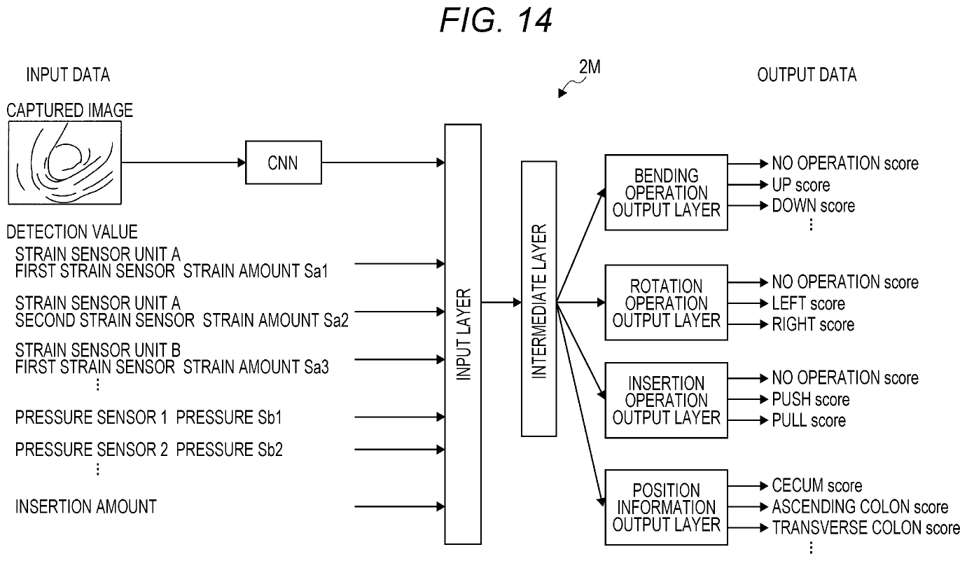

FIG. 14 is an explanatory diagram for explaining a configuration of a learning model 2M of the second embodiment. The learning model 2M in the second embodiment is, for example, a CNN. The learning model 2M includes an input layer that receives a captured image and a detection value at the same time point, an output layer that outputs operation information of a next stage and information regarding a position of the endoscope 4, and an intermediate layer (hidden layer) that extracts feature amounts of the captured image, the detection value, and an insertion amount. Note that the captured image may be an image in which feature data extracted via a network including a convolution layer is input to the learning model 2M. The input element of the learning model 2M may include the insertion amount of the endoscope 4.

The insertion amount of the endoscope 4, which is input to the input layer, is an insertion amount of the insertion portion 42 with respect to a body of the subject. The processor 2 for an endoscope includes, for example, an insertion amount detection unit (not illustrated), and detects the insertion amount of the insertion portion 42 into the subject. The insertion amount detection unit is disposed near a lumen portion (for example, an anus) of the subject into which the insertion portion 42 is inserted. The insertion amount detection unit has an insertion hole through which the insertion portion 42 is inserted, and detects the insertion portion 42 inserted through the insertion hole. The insertion amount detection unit includes, for example, a rotating body that rotates in contact with the insertion portion 42 of the endoscope 4, a rotary encoder that detects a rotation amount of the rotating body, and detects a movement amount of the insertion portion 42 in the longitudinal direction. The insertion amount detection unit may detect a magnetic coil 651 built in the insertion portion 42 by using, for example, a magnetic sensor. The processor 2 for an endoscope can calculate an insertion length of the insertion portion 42 from the insertion amount detection unit by using the detection result of the insertion amount detection unit.

For example, the information regarding the position of the endoscope 4, which is output from the output layer, is information indicating the position of the endoscope 4 in a large intestine. The output information may include sites in the large intestine, such as a cecum, an ascending colon, a transverse colon, a descending colon, a sigmoid colon, a rectosigmoid, an upper rectum, a lower rectum, an anal canal. In a case where the captured image, the detection value, the insertion amount, and the like are input, the learning model 2M is trained to output the operation information of the next stage and the information regarding the position of the endoscope 4.

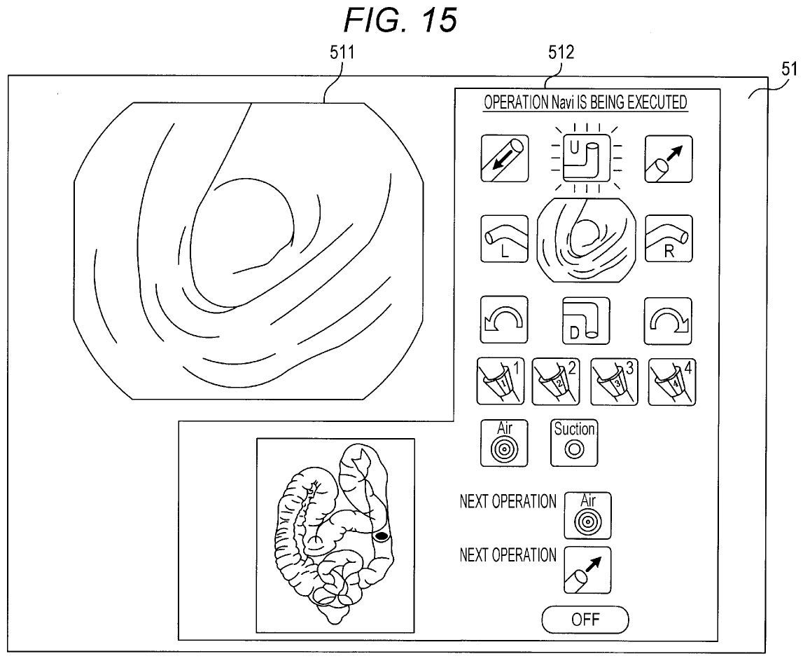

FIG. 15 is a diagram illustrating an example of a screen displayed on a display device 5. The display device 5 displays an operation information screen 51 based on screen information including the output information of the learning model 2M in the second embodiment. In the example illustrated in FIG. 15, on the operation information screen 51, an endoscopic image 511 and a navigation image 512 including the operation information of the next stage and the information regarding the position of the endoscope 4 are displayed in parallel.

The information regarding the position of the endoscope 4 is displayed, for example, by superimposing an object such as a circle indicating the position of the distal end portion 45 on an image indicating the large intestine. In a case where the site (position in the large intestine) output by the learning model 2M is specified, the control unit 21 acquires a position corresponding to the specified site with reference to a table (not illustrated) that stores position coordinates of the site and the object in association with each other. The control unit 21 performs image processing such as superimposing the object indicating the position of the endoscope 4 on the image indicating the large intestine based on the acquired position, and then displays a navigation image 512 obtained after the image processing on the display device 5. According to the present embodiment, by outputting the next operation content together with the position of the endoscope 4, more information regarding the state of the endoscope 4 and the subsequent operation content is provided to support a smooth endoscope operation of the operator.

Note that the embodiments disclosed as described above should be considered to be exemplary in all respects without being limited. The technical features described in the embodiments can be combined with each other, and the scope of the present invention is intended to include all modifications within the scope of the claims and the scope equivalent to the claims.

### REFERENCE SIGNS LIST

10 Endoscope system
2 Processor for endoscope
21 Control unit
22 Main storage device
23 Auxiliary storage device
2P Program
2M Learning model
4 Endoscope
41 Operation unit
42 Insertion portion
43 Soft portion
44 Bending portion
45 Distal end portion
5 Display device
61 Strain sensor unit
611 First strain sensor
612 Second strain sensor
62 Pressure sensor unit
621 Pressure sensor
63 Acceleration sensor
64 Angle sensor
65 Magnetic sensor
651 Magnetic coil

1. A processor for an endoscope, the processor comprising:
an acquisition unit that acquires a detection value detected by an endoscope or a captured image captured by the endoscope;
a specification unit that specifies operation information of a next stage based on the detection value or captured image acquired by the acquisition unit; and
an output unit that outputs the operation information specified by the specification unit.

2. The processor for an endoscope according to claim 1, wherein
in a case where the detection value detected by the endoscope or the captured image captured by the endoscope is input, the specification unit inputs the detection value or captured image acquired by the acquisition unit to a trained learning model so as to output the operation information of the next stage, and acquires operation information of the next stage output from the learning model.

3. The processor for an endoscope according to claim 1, wherein
the output unit outputs image data including the operation information.

4. The processor for an endoscope according to claim 1, wherein
the output unit iconifies and outputs the operation information.

5. The processor for an endoscope according to claim 1, wherein
the detection value is detected by a sensor disposed on an insertion portion of the endoscope.

6. The processor for an endoscope according to claim 5, wherein
the sensor is a pressure sensor or a strain sensor.

7. The processor for an endoscope according to claim 5, wherein
the sensor is at least any one selected from an angle sensor, a magnetic sensor, or an acceleration sensor.

8. The processor for an endoscope according to claim 1, wherein
the operation information includes at least any one of information regarding a forward and backward direction of a distal end of the endoscope, information regarding a bending direction of the distal end of the endoscope, and information regarding a rotation direction of the distal end of the endoscope.

9. The processor for an endoscope according to claim 1, wherein
the operation information includes at least any one of information regarding an air supply operation of the endoscope, information regarding a suction operation of the endoscope, or information regarding hardness of a soft portion of the endoscope.

10. An endoscope comprising an insertion portion having a strain sensor disposed on a flexible soft portion, wherein
the strain sensor includes one or a plurality of sets of sensors including a set of a first strain sensor and a second strain sensor, and
the first strain sensor and the second strain sensor are disposed at positions separated by a central angle of approximately 90 degrees on one circumference on an outer circumference of the insertion portion.

11. An endoscope system comprising an endoscope and a processor for an endoscope, wherein
the endoscope includes
an insertion portion having a strain sensor disposed on a flexible soft portion,
the strain sensor includes one or a plurality of sets of sensors including a set of a first strain sensor and a second strain sensor,
the first strain sensor and the second strain sensor are disposed at positions separated by a central angle of approximately 90 degrees on one circumference on an outer circumference of the insertion portion, and
the processor for an endoscope includes
an acquisition unit that acquires a detection value detected by the strain sensor and a captured image captured by the endoscope,
a specification unit that specifies operation information of a next stage based on the detection value and captured image acquired by the acquisition unit, and
an output unit that outputs the operation information specified by the specification unit.

12-14. (canceled)
